(19) United States
(12) Patent Application Publication
(10) Pub. No.: US 2024/0060965 A1
(43) Pub. Date: Feb. 22, 2024

(54) CAR THERAPY RESPONSE PREDICTION

(71) Applicants: ICHILOV TECH LTD., Tel-Aviv (IL); SCOPIO LABS LTD., Tel Aviv (IL)

(72) Inventors: Benzi KATZ, Tel-Aviv (IL); Irit AVIV, Tel-Aviv (IL); Shahar KARNY, Hod Hasharon (IL); Hadar SHIMONI, Modi'in (IL); Amit NATAN, Hod Hasharon (IL); Ofek AIZENMAN, Holon (IL)

(21) Appl. No.: 18/235,922

(22) Filed: Aug. 21, 2023

Related U.S. Application Data
(60) Provisional application No. 63/399,704, filed on Aug. 21, 2022.

(51) Int. Cl. G01N 33/50; G01N 1/30

(52) U.S. Cl. CPC G01N 33/5094; G01N 1/30; G01N 2800/52

(57) ABSTRACT

Methods of predicting the response of a subject to chimeric antigen receptor (CAR) therapy comprising receiving peripheral blood smears (PBS) from the subject and determining the number of CAR cells in the PBS are provided.

### CROSS REFERENCE TO RELATED APPLICATIONS

This application claims the benefit of priority of U.S. Provisional Patent Application No. 63/399,704, filed Aug. 21, 2022, the contents of which are all incorporated herein by reference in their entirety.

### FIELD OF INVENTION

The present invention is in the field of CAR-T therapy and diagnostics.

### BACKGROUND OF THE INVENTION

Chimeric Antigen Receptor T-cell (CAR-Tc) therapy becomes the preferable therapeutic approach in patients with relapsed and refractory diffuse large cell B cell non-Hodgkin lymphoma (R/R DLBCL). Tisagenlecleucel (Tisa-cel) and Axicabtagene Ciloleucel (Axi-cel) are two FDA approved CAR-Tc for the treatment of R/R DLBCL, providing durable responses in 30%-40% of patients. Consequential adverse effects include the cytokine release syndrome (CRS) reported in up to 93% of patients, with 13% to 22% of grade 3 or higher, and immune effector cell-associated neurotoxicity syndrome (ICANS) reported in up to 67% of patients, with 12% to 28% of grade 3 or higher.

Efforts to predict response to treatment and define risk factors for the development of clinically significant adverse events, focus on patient and lymphoma-related characteristics and evaluate CAR-Tc expansion post transfusion (mainly in the setting of clinical research). Currently, there are no specific recommendations or commercial assays for routine CAR-Tc measurements following transfusion and polymerase chain reaction (PCR) or flow cytometry (FC) analysis are used in clinical studies for quantitative measurements of CAR-Tc expansion and persistence.

Detection of CAR-Tc in peripheral blood smear (PBS) is challenging due to insufficient data regarding morphology prior to transfusion and low sensitivity of currently available morphological tools, being able to analyze only few snapshots of WBC, precluding a reliable analysis especially in leukopenic patients. Scopio Labs Full-Field-Morphology (FFM) is a novel digital microscopy platform that provides high-resolution images combined with wide field of view that incorporate artificial intelligence classification capabilities. The full filed/high resolution combination enables detection and classification of rare cells in PBS derived from leukopenic samples that are common during the first days following CAR-Tc administration. In this study, we first evaluated the morphology of CAR-Tc cells in the products, prior to their administration to the patients. Next, we investigated the ability of the high-resolution/full field FFM platform to detect and define CAR-Tc morphology in PBS, analyze CAR-Tc kinetics and dynamics, and predict response to treatment and the development of treatment-related adverse events (particularly CRS) in R/R DLBCL patients treated with CAR-Tc. A new accurate test for determining the patient specific effectiveness and risk of adverse effects is greatly needed.

### SUMMARY OF THE INVENTION

The present invention provides methods of predicting the response of a subject to chimeric antigen receptor (CAR) therapy comprising receiving peripheral blood smears (PBS) from the subject and determining the number of CAR cells in the PBS.

According to a first aspect, there is provided a method of predicting subject response to chimeric antigen receptor (CAR) therapy, the method comprising:
a. receiving peripheral blood smears (PBS) from the subject after the CAR therapy; and
b. determining the number of CAR cells in the PBS;
thereby predicting subject response to CAR therapy.

According to some embodiments, the CAR therapy comprises administration of CAR T cells.

According to some embodiments, the CAR is an anti-CD19 CAR.

According to some embodiments, the CAR therapy comprises the administration of Tisagenlecleucel or Axicabtagene Ciloleucel.

According to some embodiments, the subject suffers from cancer.

According to some embodiments, the cancer is a hematological cancer.

According to some embodiments, the hematological cancer is a lymphoma.

According to some embodiments, the lymphoma is diffuse large cell B cell non-Hodgkin lymphoma (DLBCL).

According to some embodiments, the DLBCL is relapsed and refractory DLBCL (R/R DLBCL).

According to some embodiments, a number of CAR cells in the PBS above a predetermined threshold indicates the subject is likely to respond to the CAR therapy.

According to some embodiments, the predetermined threshold is the number of CAR cells in PBS from subjects that do not respond to the CAR therapy.

According to some embodiments, response to the CAR therapy is complete response (CR).

According to some embodiments, the PBS was produced from peripheral blood obtained from the subject at about day 5 after the CAR therapy.

According to some embodiments, the CR is CR at day 30 or beyond after the CAR therapy.

According to some embodiments, a number of CAR cells in the PBS above a predetermined threshold indicates the subject is likely to suffer from an extended cytokine release syndrome (CRS) as compared to the average CRS duration in subjects receiving the CAR therapy.

According to some embodiments, the predetermined threshold is the number of CAR cells in PBS from subjects without an extended CRS.

According to some embodiments, the PBS was produced from peripheral blood obtained from the subject at about day 14 after the CAR therapy.

According to some embodiments, the number of CAR cells is above the predetermined threshold in PBS produced from peripheral blood obtained from the subject at at least 3 time points between days 1 and 14 after the CAR therapy.

According to some embodiments, the number of CAR cells is the total number of CAR cells.

According to some embodiments, the number of CAR cells is the total number of activated CAR cells.

According to some embodiments, the number of CAR cells is the total number of regular CAR cells.

According to some embodiments, the method further comprises administering the CAR therapy to the subject, obtaining peripheral blood from the subject and producing smears from the peripheral blood.

According to some embodiments, the determining the number of CAR cells in the PBS comprises counting the CAR cells with a digital microscopy platform.

According to some embodiments, the digital microscopy platform employs machine learning to determine cell classes based on morphology.

According to some embodiments, the digital microscopy platform is the Scopio Labs X100 Full Field PBS system.

According to some embodiments, the method further comprises administering an alternative therapy to a subject not predicted to response to the CAR therapy.

According to some embodiments, the method further comprises administering a second dose of CAR therapy to a subject predicted to respond to the CAR therapy.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B. The production process of CAR-Tc, and lymphocytes characterization. (1A) CAR-Tc production and activation timeline. Time points of activation of the cells with anti-CD3/CD28, CAR transduction, incubation with IL-2, addition of target cells and sorting/harvesting of the experiments are indicated. The average transduction percentage was 32.56% on day 7 (±2.98%, n=3). (1B) Percentage of cells expressing CD3, CD4 and CD8 at indicated days post activation as was analyzed by flow cytometry (αhCD3-APC, αhCD4-FITC, αhCD8-PB). The results are the average ±S.D. of three productions.

FIGS. 2A-2E: Pre-infusion CAR-Tc morphology. Micrographs of 5 morphologically distinct subpopulations of CART cells: (2A) quiescent CAR-T cells, (2B) activated morphology CAR-T cells, (2C) apoptotic CAR-T cells, (2D) multi-nucleated CAR-T cells, and (2E) mitotic CAR-T cells.

FIGS. 3A-3C. CAR-T cells exhibit activated morphology following specific target encounter in vitro. (3A) percentage of cells exhibiting the lymphocytes (top), quiescent (middle) and activated (bottom) morphology at indicated days post activation of transduced (white circles) or un-transduced cultures (black circles). The results are the average ±S.D. of three productions. (3B) Percentage of cells exhibiting activated morphology following co-culture with CD19+ tumor cells. At day 7, transduced or un-transduced lymphocytes were co-culture in a 2:1 effector:target ratio with the CD19 expressing Raji cell line for 16 h. Cultures was then sorted, and all CD19 negative cells, which were sorted to CAR positive or CAR negative cells, were collected. Sorted cells (sorting is shown below, cells in squares) were fixed, stained and their morphology was determined by microscopic examination. The results are the average ±S.D. of three productions. P values are presented, with p<0.05 considered significant. (3C) Representative images of cells at day 8 of the experiment, with each treatment designated at the bottom.

FIGS. 4A-4D: Acquisition and classification of CAR-Tc in PBS by FFM. Micrographs of FFM analysis showing (4A) the large area analyzed, (4B) grid of 1000 fields of vision and identification of individual white blood cells, (4C) 100× magnification of individual fields of vision containing white blood cells and (4D) a close-up identification of the cells (performed manually). Acquisition and classification of CAR-Tc in PBS by FFM. (4A). A portion of the scanned slide is shown (4B), where each square represents a 100× magnification FOV. WBC were detected (4C) and classified manually by a morphology expert according to their morphology. Examples for regular (top), activated (middle) and apoptotic (bottom) morphology CAR-Tc are shown (4D).

FIGS. 5A-5D: Correlations between CAR-Tc in PBS and response. (5A-C) Bar graphs of the number of (5A) CAR T cells of various morphologies, (5B) various white blood cells and (5C) CAR T cells at various time points in subject that eventually had or did not have complete response (CR) to the therapy. (5D) Micrograph of activated CAR-T cells at day 5. Correlations between CAR-Tc in PBS and response. (5A) Average ±S.D. of CAR-Tc subtypes/day 5 PBS was determined in patients who did (white bars) or did not (black bars) achieve complete response (CR). *=p<0.03. (5B) The average ±S.D. of basophils, eosinophils, monocytes, lymphocytes and neutrophils/day 5 PBS was determined in patients who did or did not achieve CR. NS=non-significant. (5C) The average ±S.D. of activated morphology CAR-Tc was in 5 days intervals post transfusion in patients who did or did not achieve CR. *=p<0.03. D. Typical D5 activated morphology CAR-Tc. Arrow indicates nucleoli, arrowhead points to white peri-nuclear hallow.

FIG. 6: Correlation between activated morphology CAR-Tc and CRS. Dot plot of activated CAR-T cell number from 166 consecutive PBS of 26 patients day 1 to 14 post CAR-Tc transfusion and the correlation to cytokine response syndrome (CRS) duration and onset and micrographs of the activated CAR-T cells. Correlation between activated morphology CAR-Tc and CRS. 166 consecutive PBS of 26 patients were prepared and scanned at days 1-14 post CAR-Tc transfusion. Activated morphology CAR-Tc were classified manually by morphology experts. CRS severity grading were determined according to the American Society for Transplantation and Cellular Medicine (ASTCT) consensus. Correlations between CRS onset (black circles) or CRS duration (white circles) and average number of activated morphology CAR-Tc/PBS were determined during days 1-14. *=p<0.03 (left). Typical day 14 activated morphology CAR-Tc are shown (right), with multiple nucleoli and peri-nuclear white hallow in some of the cells.

FIGS. 7A-7C: CAR-Tc morphology during the production process. (7A) Micrograph of Day 1. The cultures contain mostly normal morphology lymphocytes, and the rest were predominantly monocytes. (7B) Micrograph of Day 7. Un-transduced cultures. Most of the cells exhibited a quiescent morphology. (7C) Micrographs of Day 8. Sorted CAR+ cells that were incubated with target cells. Conjugates between activated morphology CAR-Tc and apoptotic cells were observed (arrows), with granules in the CAR-Tc concentrating within the conjugation area (top images, inserts).

FIG. 8: A line graph of the average number of cells per PBS showing total white blood cells and total CAR T cells. Post-transfusion total WBC and CAR-Tc quantification in PBS. 166 consecutive PBS of 26 patients were prepared and scanned at days 1-14 post CAR-Tc transfusion. Both total WBC and CAR-Tc were classified manually by morphology experts. The average dynamics of total WBC (red line) and of CAR-Tc (blue line) is presented.

### DETAILED DESCRIPTION OF THE INVENTION

The present invention, in some embodiments, provides methods of predicting the response of a subject to chimeric antigen receptor (CAR) therapy comprising receiving peripheral blood smears (PBS) from the subject and determining the number of CAR cells in the PBS.

By a first aspect, there is provided a method of predicting subject response to chimeric antigen receptor (CAR) therapy, the method comprising receiving peripheral blood smears (PBS) from the subject and determining the number of CAR cells in the PBS, thereby predicting subject response to CAR therapy.

In some embodiments, the method is an in vitro method. In some embodiments, the method is an ex vivo method. In some embodiments, the method is a computerized method. In some embodiments, the method is a diagnostic method. In some embodiments, the method is a prognostic method. In some embodiments, predicting response is determining response. In some embodiments, predicting response is determining likely response. In some embodiments, predicting response is determining response probability.

In some embodiments, CAR therapy comprises administration of CAR cells to the subject. In some embodiments, CAR cells are cells that express the CAR. In some embodiments, express is expresses in the plasma membrane. CAR therapy is well known in the art and any CAR cells may be used for the method of the invention. In some embodiments, the CAR cells are CAR T cells. In some embodiments, the CAR cells are CAR NK cells. In some embodiments, the CAR cells are CAR T or NK cells. In some embodiments, the administration is intravenous administration. In some embodiments, the CAR is an anti-CD19 CAR. In some embodiments, the CAR binds to CD19. Anti-CD19 CARs are well known in the art and any such CAR may be used. In some embodiments, the CAR therapy comprise administration of Tisagenlecleucel. In some embodiments, the CAR therapy comprise administration of Axicabtagene Ciloleucel. In some embodiments, the CAR therapy comprise administration of Tisagenlecleucel, Axicabtagene Ciloleucel or a combination thereof.

In some embodiments, the subject is a mammal. In some embodiments, the mammal is a human. In some embodiments, the subject suffers from cancer. In some embodiments, the cancer is a hematological cancer. In some embodiments, the cancer is a blood cancer. In some embodiments, the cancer is lymphoma. In some embodiments, the lymphoma is non-Hodgkin lymphoma. In some embodiments, the cancer is diffuse large cell B cell non-Hodgkin lymphoma (DLBCL). In some embodiments, the cancer is primary mediastinal large B-cell lymphoma (PMBCL). In some embodiments, the cancer is relapsed and refractory diffuse large cell B cell non-Hodgkin lymphoma (R/R DLBCL).

In some embodiments, the PBS is from the subject after CAR therapy. In some embodiments, the PBS is produced from peripheral blood (PB) from the subject. In embodiments, the PB is obtained from the subject at about 5 days after the CAR therapy. In some embodiments, after the CAR therapy is after administration of the CAR therapy. In some embodiments, after the CAR therapy is after injection of the CAR cells. In embodiments, the PB is obtained from the subject at about 14 days after the CAR therapy. In embodiments, the PB is obtained from the subject at a plurality of time points from 1 to 14 days after the CAR therapy. In some embodiments, a plurality is at least 2, 3, 4, 5, 6, 7, 8, 9, 10, 11, 12, 13 or 14 time points. Each possibility represents a separate embodiment of the invention. In some embodiments, a plurality is at least 3 times. In some embodiments, samples are taken continuously from day 1 to 14. In some embodiments, continuously is each day. In some embodiments, the method further comprises obtaining PB from the subject. In some embodiments, obtaining is extracting. In some embodiments, the method further comprises producing smears from the PB.

In some embodiments, a number of CAR cells in the PBS above a predetermined threshold indicates the subject is likely to respond to the CAR therapy. In some embodiments, a subject likely to respond is a subject likely to display response. some embodiments, a subject likely to respond is a subject that responds. In some embodiments, a subject likely to respond is a responder. In some embodiments, response is complete response (CR). In some embodiments, response is partial response (PR). In some embodiments, response is CR or PR. In some embodiments, non-response is not CR. In some embodiments, non-response is stable disease (SD) or progressive disease (PD). In some embodiments, non-response is PD. In some embodiments, non-response is not CR or PR. It will be understood by a skilled artisan that response is a clinical parameter that can be determined by any known scale of determining response. In some embodiments, response is response at 30 days after CAR therapy. In some embodiments, after CAR therapy is after CAR administration.

In some embodiments, a number of CAR cells in the PBS above a predetermined threshold indicates the subject is likely to suffer from an extended cytokine release syndrome (CRS). In some embodiments, a subject likely to suffer is a subject that does suffer. In some embodiments, extended is as compared to the average CRS duration. In some embodiments, the average CRS duration is the average in subject receiving the CAR therapy.

In some embodiments, the predetermined threshold is the number of CAR cells in PBS from subjects that do not respond to the CAR therapy. In some embodiments, the number is the average number. In some embodiments, the threshold is a number statistically significantly different from the number in PBS from subjects that do not respond to the CAR therapy. In some embodiments, the predetermined threshold is the number of CAR cells in PBS from subjects without extended CRS.

In some embodiments, the number of CAR cells is the total number of all CAR cells. In some embodiments, all CAR cells comprise all cells expressing said CAR. In some embodiments, all CAR cells comprise regular CAR cells, activated CAR cells, apoptotic CAR cells, multinucleated CAR cells and mitotic CAR cells. In some embodiments, the number of CAR cells is the total number of regular CAR cells. In some embodiments, the number of CAR cells is the total number of activated CAR cells. In some embodiments, the subtype of CAR cells is determined by morphology. Morphological characteristics of the various subtypes are as described hereinbelow.

In some embodiments, the determining the number of CAR cells in the PBS comprises counting the CAR cells. In some embodiments, the determining is by digital microscopy. In some embodiments, digital microscopy is a digital microscopy platform. In some embodiments, the digital microscopy platform employs machine learning (ML) to determine cell type/class. In some embodiments, the ML is an ML algorithm. In some embodiments, the ML is artificial intelligence. In some embodiments, the ML comprises a classifier. In some embodiments, the ML classifies cells by cell type or class. In some embodiments, the classification is based on morphology. In some embodiments, the ML is trained on cells of different morphologies. In some embodiments, the ML determines a cell is a white blood cell (WBC). In some embodiments, a WBC is a lymphocyte. In some embodiments, the ML determines a cell is a CAR cell. In some embodiments, the ML determines the subtype of the CAR cell. In some embodiments, the subtype of the CAR cell is determined manually. In some embodiments, the digital microscopy platform in a full field morphology (FFM) system. In some embodiments, the FFM system is the Scopio Labs X100 Full Field PBS system.

In some embodiments, the method further comprises administering said CAR therapy to the subject. In some embodiments, the administering is before the obtaining peripheral blood. In some embodiments, the administering is before the receiving PBS. In some embodiments, the method further comprises administering an alternative therapy to a subject not predicted to response to the CAR therapy. In some embodiments, a subject not predicted to respond is a subject predicted not to respond. In some embodiments, the method further comprises administering a second dose of CAR therapy to a subject predicted to respond to the CAR therapy.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

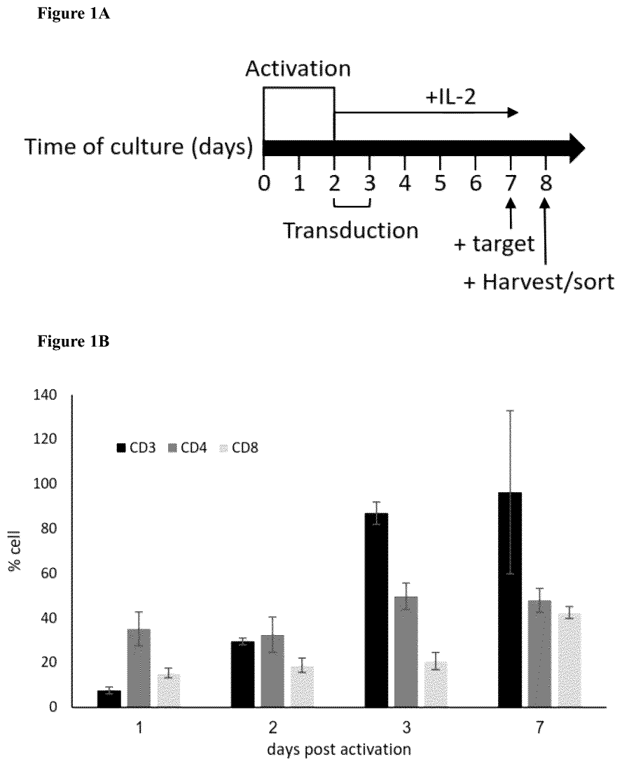

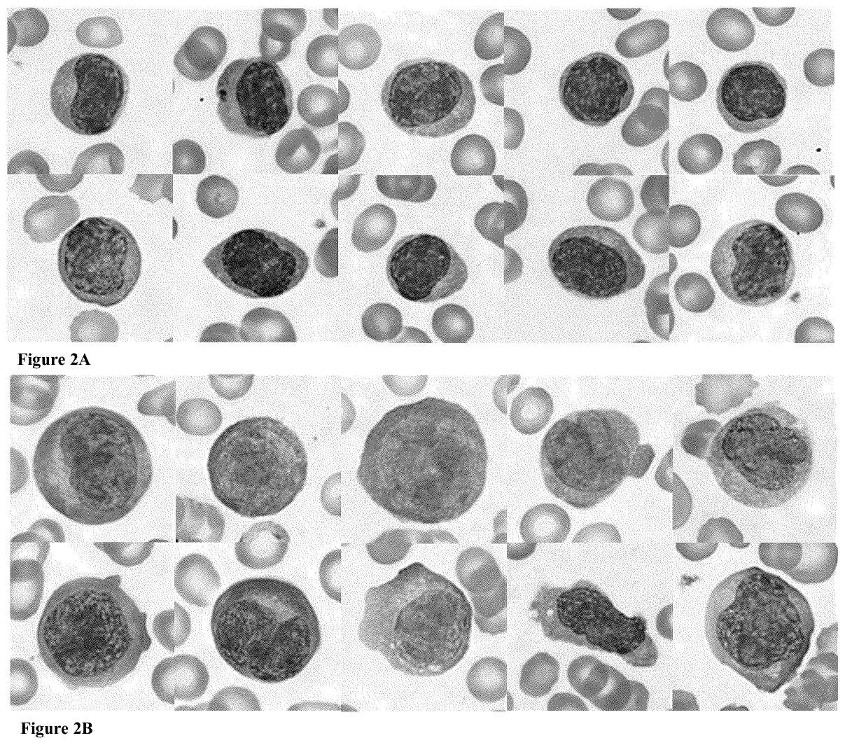

In vitro generation and activation of CAR-Tc: Peripheral blood mononuclear cells (PBMC) from three independent healthy donors were subjected to CAR-Tc production process described in FIG. 1A. T cell transduction: retroviral transduction of T cells was performed as previously described17,18. Briefly, peripheral human blood lymphocytes (PBL) were isolated from the blood of healthy human donors by density gradient centrifugation on Ficoll-Paque (Axis-shield). PBLs were activated in nontissue culture treated 6-well plates, precoated with anti-human CD3 and anti-human CD28 for 48 hours at 37° C. Activated lymphocytes were harvested and subjected to two consecutive retroviral transductions in RetroNectin (Takara; T100B) precoated, nontissue culture—treated 6-well plates supplemented with human IL-2 (Novartis-Pharma; 4764111 U57, 100 IU/mL). After transduction, cells were cultured in the presence of 350 IU/mL IL2. Target engagement of CAR-T cells: CAR-T cells were co-cultured with target cells (Raji cell line, Kindely provided by Tova Wax, Weizmann Institute of Science) expressing CD19 against which the CAR is directed. The effector: target ratio was 2:1. the cells were co-cultured for 16 h in RPMI and a concentration of 1×106 lymphocytes/ml. cells were then collected, stained against either CD19 (to detect target cells) or CAR and sorted accordingly. At various stages of the production process cells were harvested, applied to glass slides (SP-Slides, Sysmex, Lincolnshire, IL) and stained by the May Grunwald/Giemsa Staining protocol described below. The slides were scanned by a Scopio Labs X100 scanner (Scopio Labs), and subjected to morphological analysis.

Flow cytometry analysis and sorting: Anti hCD3-APC (300439), anti hCD4-FITC (300506), anti hCD8-PB (301023) and streptavidin-APC (405207) all purchase from BioLegend (London, UK). Rabbit Anti-CAR antibody was prepared in house cleaned and biotinylated. Cell surface markers as well as percentage of lymphocytes transduction were analyzed by flow cytometry. Cells were incubated on ice for 30 min with the appropriate antibody. For CAR detection a 2-step staining protocol was used. First, a 30 min incubation with the biotinylated anti-CAR antibody followed by 30 min incubation with streptavidin-APC. Staining and cell washes were done with 0.1 ml and 2 ml FACS buffer respectively (2% FCS, 0.05% sodium azid, 2 mM EDTA in PBS). Cells were then analyzed by FACSCantoII flow cytometer (Becton Dickinson, San Jose, CA). Data analysis was carried out using FCS Express software. For sorting, cells were labeled with the designated antibodies as described above, and were sorted with FACSAria III (Becton Dickinson, San Jose, CA).

Preparation of slides from CAR-Tc products: Attempts to prepare slides directly from the CAR-Tc products, even when diluted into several types of media, phosphate buffered saline, phosphate buffered saline with 5% bovine serum albumin, RPMI tissue culture medium, without or with fetal calf serum, have all failed to provide high quality samples adequate for morphological analysis. Hence, leftovers of CAR-Tc transfusion bags (n=3) were mixed with normal PB in 1:3 ratio, aiming to preserve CAR-Tc morphology in their natural environment, in a similar manner to a previous report19. Slides were than prepared from the CAR-Tc/PB mixture.

Patient Cohort: Twenty-six patients with R/R DLBCL receiving either Axi-cel or Tisa-cel from October 2019 through October 2020 in Tel Aviv Sourasky Medical Center (TASMC) were included. Patients underwent a PET-CT scan one month post CAR-Tc infusion, and response was defined according to the Lugano criteria. CRS and ICANS grading were determined according to the American Society for Transplantation and Cellular Medicine (ASTCT) consensus. Patient and lymphoma-related characteristics at diagnosis and at CAR-Tc administration, adverse events following CAR-Tc transfusion and response to therapy, were collected from patient's medical records. The study was approved by the local institutional review boards according to the declaration of the Helsinki accord.

Peripheral blood smears preparation and staining: Peripheral blood samples were collected into a spray-dried K3 EDTA 3.6 ml vacuum tube (Greiner, Kremsmunster, Austria). Peripheral blood smears (PBS) were prepared, within four hours at room temperature, by Beckman Coulter Slide Maker Stainer (Brea, CA, USA), on glass slides (SP-Slides, Sysmex, Lincolnshire, IL). Staining protocol: Methanol (absolute, Bio-Lab Ltd., Jerusalem, Israel): 4 minutes 1 second; May Grunwald Stain (TruColor, Beckman Coulter, Brea, CA, USA): 6 minutes 22 seconds; Water (double distilled, Terion, Cary, NC): 3 minutes; Giemsa Stain (TruColor, Beckman Coulter, Brea, CA, USA): 9 minutes; Water (double distilled, Terion, Cary, NC): 1 minutes; Air dry: 6 minutes. All slides were covered with Stisse Cover Glasses, by using EUKITT® Classic Mounting Medium Glue.

Morphological analysis: Scopio Labs FFM locates optimal analysis area for each PBS, to include the monolayer area and the feathered edge. On average, the monolayer part of the scan is 0.38 cm2, equivalent to 1000 high power fields (100× magnification). Then, Scopio Labs FFM performs white blood cell (WBC) analysis by an artificial intelligence-based classifier, in a decision support system (DSS) mode, and the resulting WBC differentials were approved (or modified where required) by qualified specialists. Alternatively, PBS were analyzed by the CellaVision DM1200 system (Lund, Sweden) according to the manufacturer's instructions in a DSS mode.

Flow Cytometry of Day 7: Clinical flow cytometry was performed on peripheral blood cells from patient at day 7 after CAR-T cells infusion. Cells were stained with the combination of 10 μg/ml FITC-labeled Human CD19 (20-29) protein Fc-Tag (Acrobiosystems Inc., Newark, DE, USA) and CD3-APCH7 (BD Biosciences, San Diego, CA, USA). Samples were acquired by FACS Cantoll and analyzed by the DIVA software (BD Biosciences, San Diego, CA, USA).

Laboratory Data: Laboratory results, including complete blood counts (CBC) and differential, CRP, LDH and ferritin were obtained from patient's medical records.

Statistics: Statistical analyses were performed with SPSS software (Version 27, Armonk, NY: IBM Corp. USA). A two tailed p value <0.05 was considered statistically significant. We used Mann-Whitney test to compare continuous variables between categories. Spearman's rank correlation coefficient was used to quantify the association between continuous variables. Friedman and Wilcoxon tests were used to compare results between periods and two types of platforms (FFM and CellaVisiom).

### Results

### Example 1: Patient Characteristics

Twenty-six R/R DLBCL patients (12 males), were included in the study. Median age at CAR-Tc transfusion was 71 years (20-84). Seventeen patients entered CAR-Tc with stable/progressive disease. Twenty-one (80.7%) patients received Tisa-cel and 5 (19.2%) received Axi-cel. Eighteen patients (69.2%) developed CRS (3 (11.5%) grade ≥3) and 7 (36.9%) developed ICANS (2 (7.69%) grade ≥3). Median durations to CRS and ICANS from infusion were 3 and 6 days respectively. PET scan performed at one month post CAR-Tc infusion revealed that 13 patients (50%) attained a complete metabolic response, 5 (19.2%) obtained partial response and 8 (30.7%) had stable or progressive disease (Patient characteristics are presented in Table 1).

TABLE 1
Baseline Characteristics, Outcome and Adverse Events of Patients

| | | Patients By product | |
|---|---|---|---|
| Variable | All Patients | Tisagenlecleucel | Axicabtagene Ciloleucel |

Characteristics at CAR-T Transfusion

| No. of patients | 26 | | 21 | | 5 | |
|---|---|---|---|---|---|---|
| Disease type - no. (%) | | | | | | |
| DLBCL | 23 | (88.5) | 19 | (90) | 4 | (80) |
| PMBCL | 3 | (11.5) | 2 | (7.7) | 1 | (20) |
| Age - Median (range) - yr. | 71 | (20-84) | 71 | (20-84) | 72 | (27-74) |
| Gender (Male) - no. (%) | 12 | (46.1) | 11 | (52.3) | 1 | (20) |
| IPI Score - no. (%) | | | | | | |
| 0-2 | 5 | (19.2) | 5 | (23.8) | 0 | (0) |
| 3-4 | 21 | (80.7) | 16 | (76.1) | 5 | (100) |
| Prior therapies - no. (%) | | | | | | |
| 1-2 | 17 | (65.3) | 15 | (71.4) | 2 | (40) |
| ≥3 | 9 | (34.6) | 6 | (28.5) | 3 | (60) |
| ECOG status score of 1 - no. (%) | 11 | (42.3) | 10 | (47.6) | 1 | (20) |
| LDH (U/L) before infusion, | 402.5 | (215-3636) | 401 | (215-3636) | 421 | (372-553) |

Median (range)

Response and Adverse events following CAR-T Treatment

| Response at one month - no. (%) | | | | | | |
|---|---|---|---|---|---|---|
| CR/PR | 13 (50)/5 (19.2) | | 10 (47.6)/4 (19) | | 3 (60)/1 (20) | |
| SD/PD | 0 (0)/8 (30.7) | | 0 (0)/7 (33.3) | | 0 (0)/1 (20) | |
| CRS - no. (%) | | | | | | |
| Any grade | 18 | (69.2) | 15 | (71.4) | 3 | (60) |
| Grade 1-2 | 15 | (57.6) | 12 | (57.1) | 3 | (100) |
| Grade ≥3 | 3 | (11.5) | 3 | (14.2) | 0 | (0) |
| ICANS - no. (%) | | | | | | |
| Any grade | 7 | (26.9) | 5 | (23.8) | 2 | (40) |
| Grade 1-2 | 5 | (19.2) | 2 | (15.3) | 2 | (40) |
| Grade ≥3 | 2 | (7.69) | 3 | (14.2) | 0 | (0) |

The abbreviation CR denotes complete response, CRS cytokine release syndrome, DLBCL diffuse large B-cell lymphoma, ECOG eastern cooperative oncology group, ICANS immune effector cell-associated neurotoxicity syndrome, IPI international prognostic index, PD, progressive disease, PMBCL primary mediastinal B-cell lymphoma, PR partial response, SD stable disease.

### Example 2: The Diverse CAR-Tc Morphology

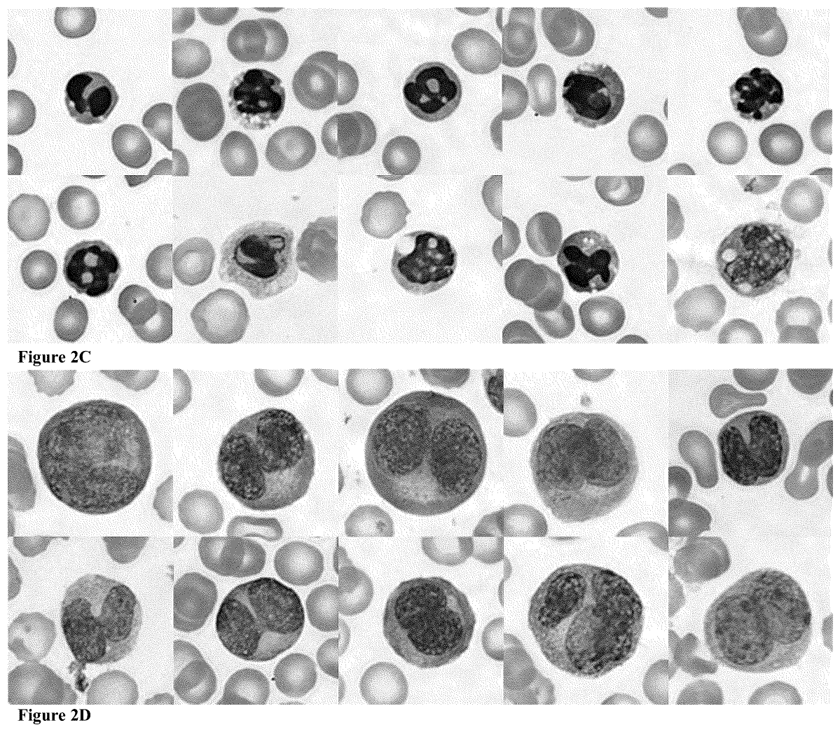

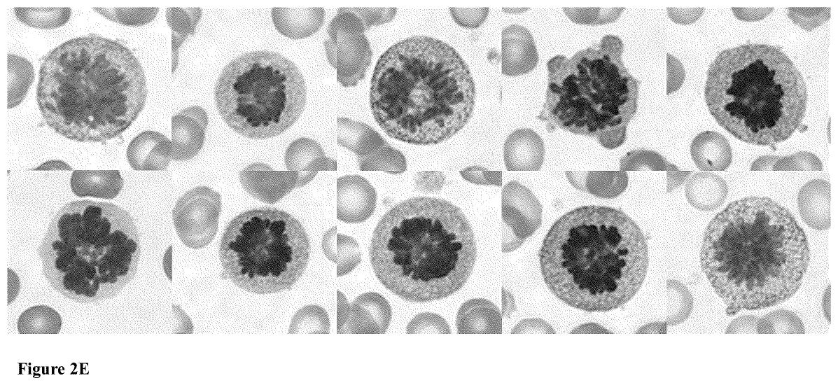

Since the morphology of CAR-Tc prior to transfusion was not yet established, we first created a basal CAR-Tc morphological library. Attempts to prepare slides directly from the CAR-Tc products, even when diluted into several types of media, phosphate buffered saline, phosphate buffered saline with 5% bovine serum albumin, DMEM tissue culture medium, without or with fetal calf serum, have all failed to provide high quality samples adequate for morphological analysis. Hence, leftovers of CAR-Tc transfusion bags (n=3) were mixed with normal peripheral blood (PB) in 1:3 ratio, aiming to preserve CAR-Tc morphology in their target environment. Slides were than prepared from the CAR-Tc/PB mixture. A total of 5,367 cells (average of 1,789 per smear, range 59-3,411), obtained from three product smears (2 Tisa-cel, 1 Axi-cel) were used to characterize basal CAR-Tc morphology. Five morphologically distinct subpopulations were identified: (1) Regular morphology CAR-Tc, small lymphocytes with basophilic cytoplasm (FIG. 2A); (2) Activated CAR-Tc, large cells with abundant basophilic cytoplasm and reticulated nucleus, with or without nucleoli (characteristics which are typical for reactive lymphocytes) (FIG. 2B); (3) Apoptotic cells, small lymphocytes with pycnotic nuclei, often with vacuoles (FIG. 2C); (4) Multinucleated CAR-Tc (FIG. 2D); and (5) mitotic CAR-Tc, referring to cells in mitosis, mostly in anaphase (FIG. 2E).

### Example 3: Characterization of CAR-Tc Morphology During their Production Process

In order to validate morphology as a reliable tool to detect, measure and follow CAR-Tc in PBS, we characterized the morphology of the cells during the various stages of the production process.

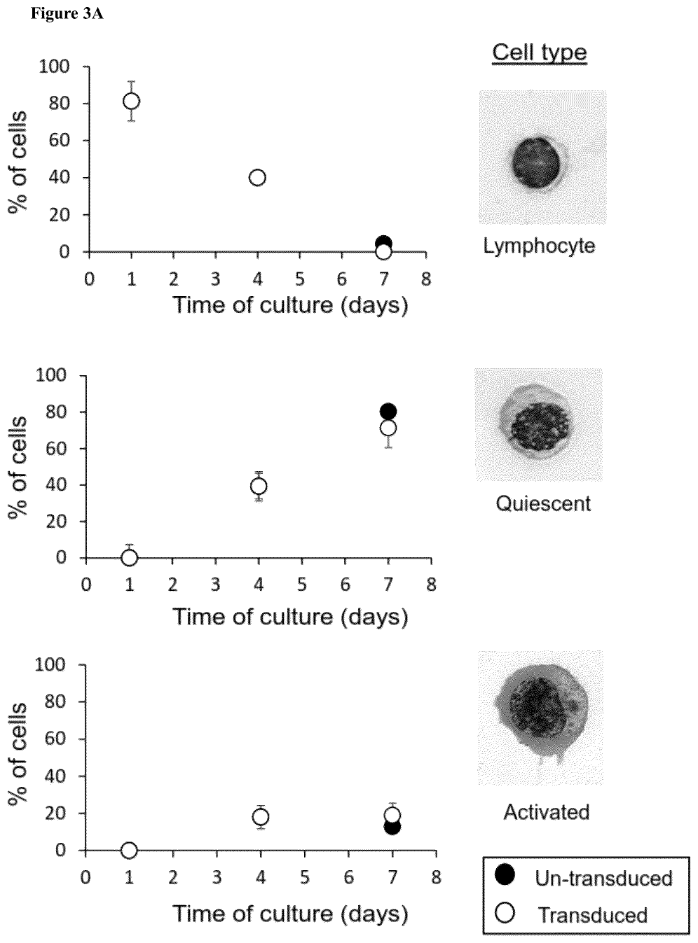

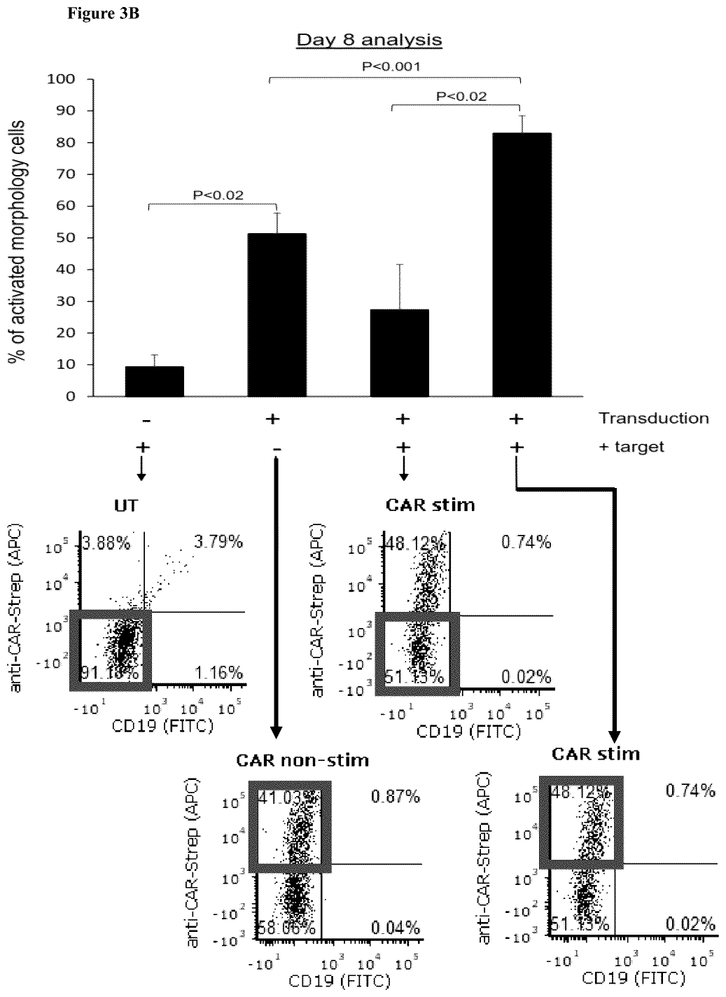

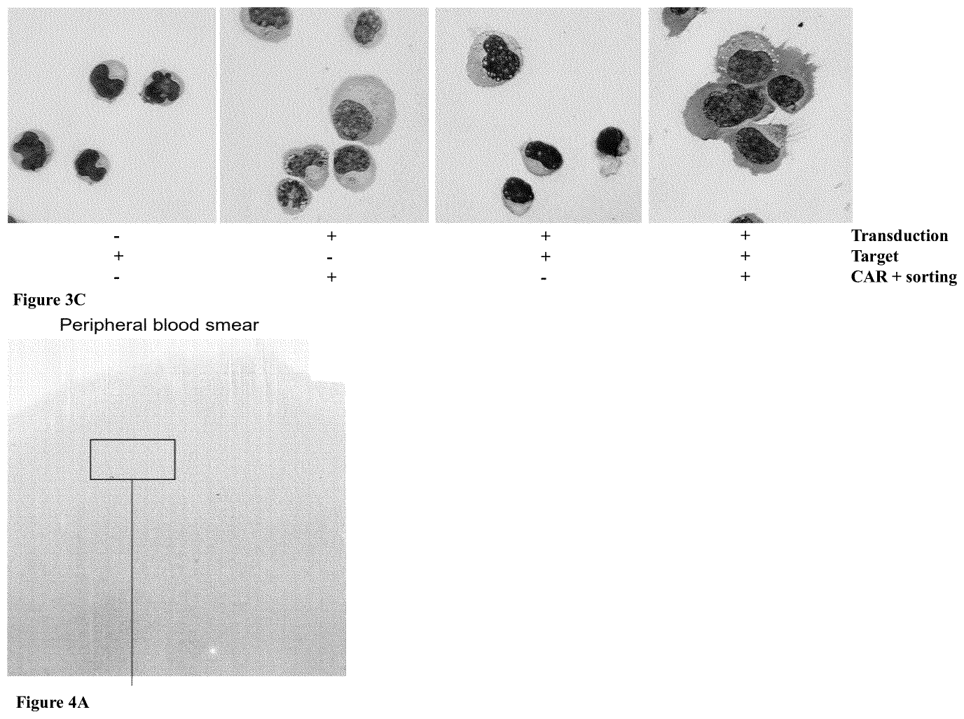

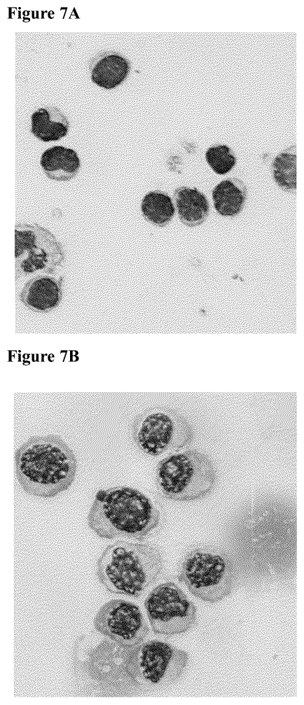

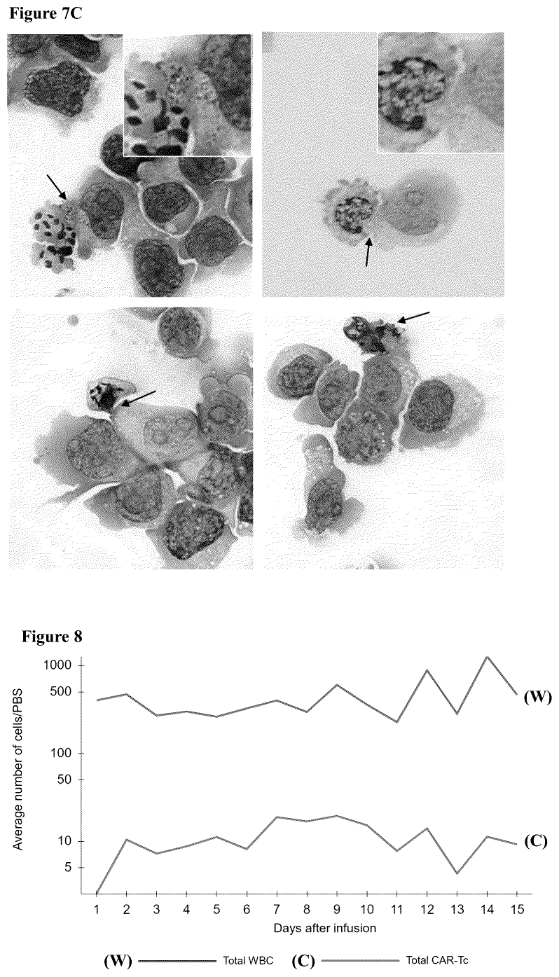

At day 1, the cultures contained 81.3±5.0% normal morphology lymphocytes, and the rest were predominantly monocytes (FIGS. 3A, and 7A). Of these cells only 7.7±1.5% were CD3+ T-cells with CD4/CD8 ratio of 2.27 (FIG. 1B). The cells were then subjected to CD3/CD28 activation (days 1-2) and transduction (days 2-3). IL-2 was added to the cultures during days 2-7 (FIG. 1A). Un-transduced cells served as controls. At day 4, 40.0±11.0% of the cells retained normal lymphocytes morphology, while 39.3±7.4% of the cells were with quiescent CAR-Tc morphology, and the rest predominantly normal morphology lymphocytes (FIG. 3A). 87.04±5.0% of these cells were CD3+ T-cells, with CD4/CD8 ratio of 2.39 (FIG. 1B). On day 7, no lymphocytes with normal morphology were observed among the transduced cells, with 71.3±7.0% of quiescent and 19.0±1.7% of activated CAR-Tc morphologies (FIG. 3A). In the un-transduced cultures of day 7, 80.3±8.1% of quiescent and 13±6.2% of activated CAR-Tc morphologies, as well as 4.3±2.3% of lymphocytes with normal morphology, were observed (FIGS. 3A, and 7B). In the transduced cultures, 96.38±1.56% were CD3+ T-cells, with 1.13 CD4/CD8 ratio (FIG. 1B). Low percentages of apoptotic cells (1.2-2.6%) appeared in the different cultures at days 0-7, while mitotic cells (0.6-7%) were observed in cultures of days 4 and 7, and only solitary bi-nucleated cells were observed (not shown).

### Example 4: Assessment of CAR-Tc Morphological Significance

At day 7, transduced or un-transduced cultures were incubated for 24 hours with CD19+ Raji B-cell line that served as target cells. At day 8, the various cells were sorted for CAR+ or CAR− expression. As shown in FIG. 3B, only 9.3±3.8 of the un-transduced cells incubated with target cells exhibited activated morphology, while most of the cells were small, with relatively dense nuclei (FIG. 3C). 42.7±10.8 of the CAR+ cells sorted from the transduced population that was not exposed to target cells had an activated morphology (FIG. 3B-3C). 27.3±14.3 CAR− cells sorted from the transduced population that was exposed to target cells had an activated morphology (FIG. 3B-3C). However, 83±5.6 CAR+ cells sorted from the transduced population that was exposed to target cells had an activated morphology, significantly higher than all the other groups (FIG. 3B-3C). Interestingly, in the sorted CAR+ cell population that were incubated with target cells, conjugates between activated morphology CAR-Tc and apoptotic cells were observed, with granules in the CAR-Tc concentrating within the conjugation area (FIG. 7C, inserts).

### Example 5: Evaluation of CAR-Tc Morphology in PBS Following Infusion

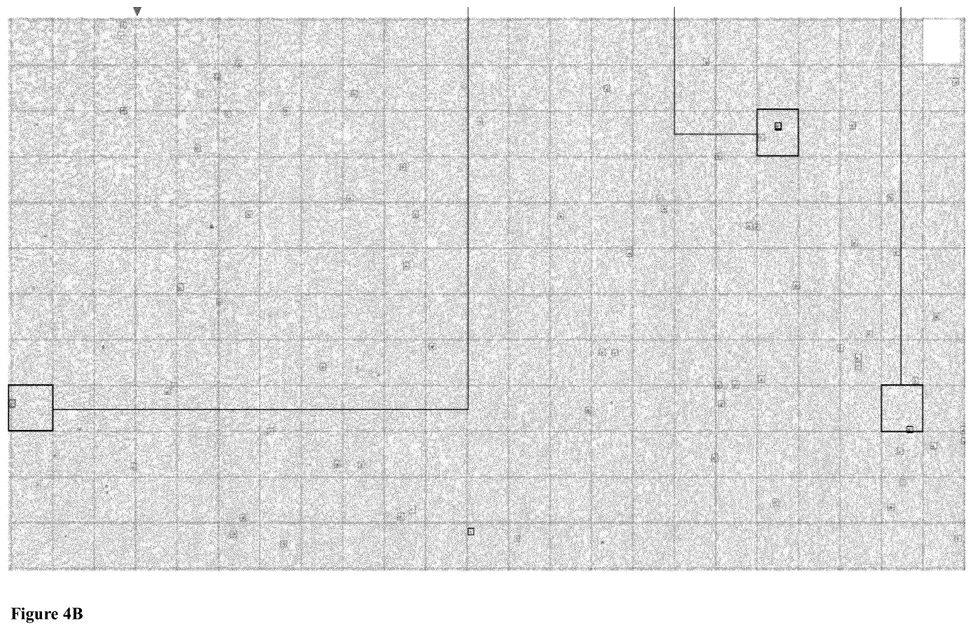

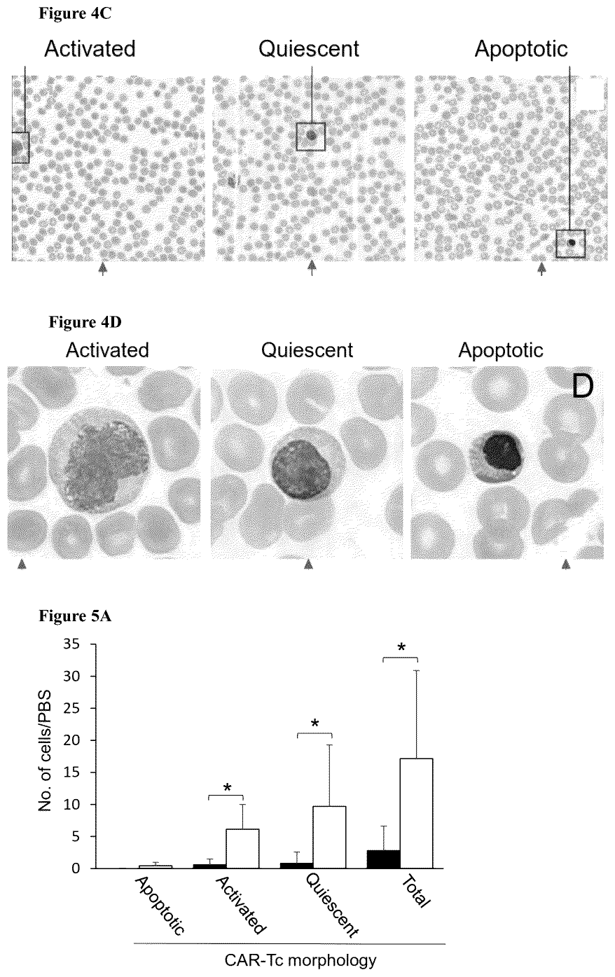

The capacity of FFM analysis to retrieve high number of cells even from leukopenic samples is demonstrated in FIG. 4. First, a large area of the PBS was scanned containing an average of 1000 fields of view (FOVs) (FIG. 4A). Then, individual WBC were detected throughout the scan (FIG. 4B). Each square represents a 100× magnification single FOV (FIG. 4B-C). Identification of cells in each PBS was determined manually (FIG. 4D). In order to compare the performance of current digital technologies, Scopio Labs FFM vs the CellaVision DM1200 platforms, 77 blood smears obtained from 18 patients were examined by both technologies. Overall, 24,247 WBC were detected by the Scopio Labs FFM platform (average 303 cells per PBS, range 0-2,295), compared with 4,335 detected by CellaVision DM1200 system (average 54.4 cells per PBS, range 0-146). During the first 4 days post-infusion, when the patients are leukopenic (<1×10\hat{ }3 WBC/ml), the CellaVision DM1200 system could hardly detected any CAR-Tc in PBS, with a median number of 2 cells only (IQR 1-2) compared with 5 (IQR 2-9) when using Scopio Labs FFM platform. Scopio Labs FFM provided a higher number of CAR-Tc in 74% (n=57) of the PBS, an identical number of CAR-Tc in 16.8% (n=13), and a lower number of CAR-Tc in 9% (n=7) of the PBS. Overall, the difference between the two methods was significant with a superior detection rate of the Scopio Labs FFM technology (median difference 2, IQR 0-6, p value <0.001).

One hundred sixty-six consecutive PBS, collected from 26 patients on days 1-15 following CAR-Tc transfusion, were analyzed by the FFM platform. Automated detection and classification of CAR-Tc into the 5 described categories, were performed, and results were validated manually by two independent hemato-pathology experts (FIG. 4). Multinucleated and mitotic CAR-Tc were scarce, and therefore withdrawn from final analysis. The average total WBC number/PBS was 453.7±280.8 (range 262-1270), relatively stable along days 1-15 following administration. The average number of total CAR-Tc/PBS was 11.0±5.0, (range 2.5-19.4) and remained relatively stable throughout days 2-15 (FIG. 8). Overall, as shown in FIG. 8, CAR-Tc accounted for a very small proportion of the total WBC in the PBS, with an average percentage of 2.8±1.4, (range 0.63%-5.7%).

### Example 6: Association Between CAR-Tc in PBS and Patient's Outcomes

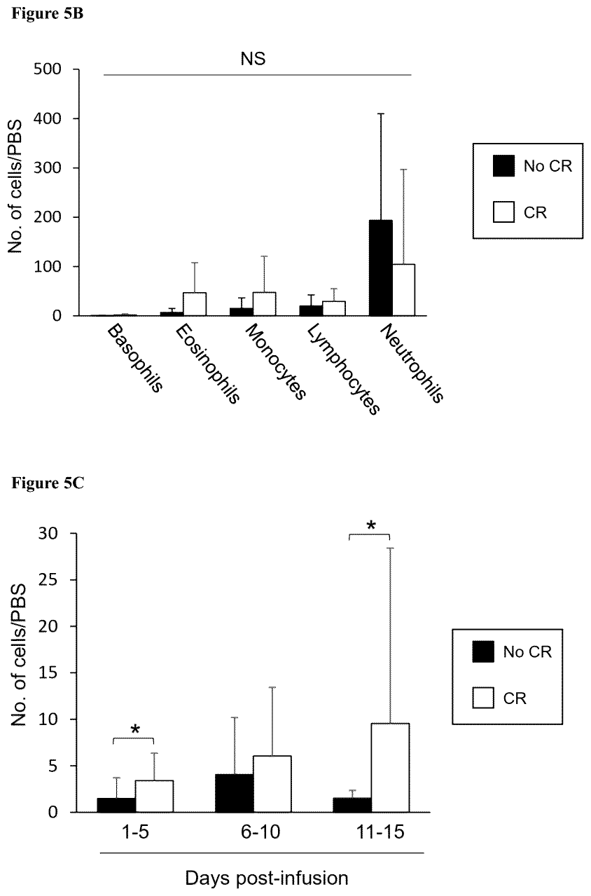

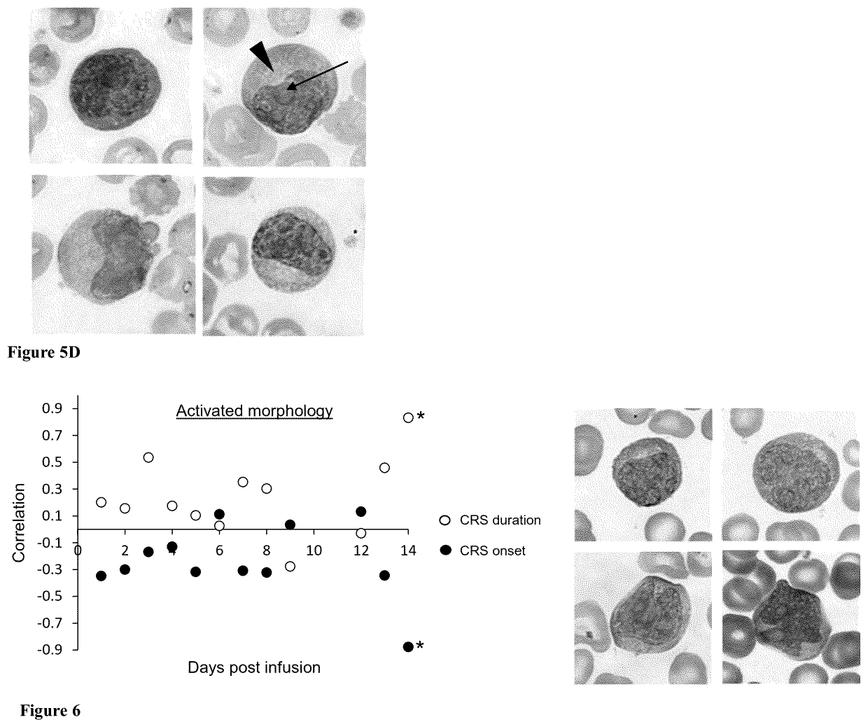

The average number of total CAR-Tc, measured on D5 post transfusion, was significantly (p=0.018) higher in patients that obtained complete response at day 30 post CAR-Tc, reaching 17.1±13.7 cells/PBS (range 2-38) vs 2.8±3.4 cells/PBS (range 0-9) for patients that did not reach CR (FIG. 5A). D5 regular and activated CAR-Tc were particularly higher in patients attaining CR: 9.7±9.6 cells/PBS (range 0-25) and 6.1±3.8 cells/PBS (range 2-12), respectively in patients with CR, vs 0.8±1.8 cells/PBS (range 0-4) and 0.6±0.9 cells/PBS (range 0-2) in patients without CR (p=0.023 and p=0.005, respectively) (FIG. 5A). No such correlation was found with other leukocyte type in the PBS (FIG. 5B). In line with that, presence of CAR-Tc in PB, assessed by measuring the average number of activated CAR-Tc on D1-5 and D11-15, was also associated with increased CR rates; average numbers of activated morphology CAR-Tc were 3.4±2.9 cells/PBS, (range 0.75-9, days 1-5), and 9.5±18.9 cells/PBS (range 0-56, days 11-15) in patients with CR, vs 2.2±0.9 cells/PBS (range 0-7, days 1-5) and 1.5±0.9 cells/PBS (range 0-3, days 11-15) in patients that did not reach CR (p=0.03 and p=0.04 respectively) (FIG. 5C). The activated CAR-Tc, measured on day 5, were large cell with abundant basophilic cytoplasm and reticulated nucleus, with (FIG. 5D, arrow) or without nucleoli, and evident peri-nuclear hallow representing developed Golgi in some of the cells (FIG. 5D, arrowhead). As expected, disease burden was also associated with outcome; patients entering CAR-Tc therapy with responsive disease (n=17) had a greater chance to achieve CR following transfusion, compared with their non-responding counterparts (n=9), (p=0.015).

### Example 7: Association Between CAR-Tc in PBS and Adverse Events

We examined the association between CAR-Tc in PBS and the occurrence of adverse events. There were no statistically significant associations between specific morphological subtypes and CRS development. However, a continues detection of activated morphology CAR-Tc thorough days 1 to 14 tended to be associated with longer duration of CRS (FIG. 6). Large numbers of activated morphology CAR-Tc in day 14 PBS were significantly correlated with long CRS, and negatively correlated with early CRS onset (FIG. 6). ICANS development was scarce in our cohort, not enabling an analysis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of predicting subject response to chimeric antigen receptor (CAR) therapy and treating said subject, the method comprising:
a. receiving peripheral blood smears (PBS) from said subject after said CAR therapy;
b. determining the number of CAR cells in said PBS, wherein a number of CAR cells in said PBS above a predetermined threshold indicates said subject is likely to respond to said CAR therapy; and
c. administering an alternative therapy to a subject not predicted to response to said CAR therapy or administering a second dose of CAR therapy to a subject predicted to respond to said CAR therapy;
thereby predicting subject response to CAR therapy and treating said subject.

2. The method of claim 1, wherein said CAR therapy comprises administration of CAR T cells.

3. The method of claim 1, wherein said CAR is an anti-CD19 CAR.

4. The method of claim 1, wherein said CAR therapy comprises the administration of Tisagenlecleucel or Axicabtagene Ciloleucel.

5. The method of claim 1, wherein said subject suffers from cancer.

6. The method of claim 5, wherein said cancer is a hematological cancer.

7. The method of claim 6, wherein said hematological cancer is a lymphoma.

8. The method of claim 7, wherein said lymphoma is diffuse large cell B cell non-Hodgkin lymphoma (DLBCL).

9. The method of claim 8, wherein said DLBCL is relapsed and refractory DLBCL (R/R DLBCL).

10. The method of claim 1, wherein said predetermined threshold is the number of CAR cells in PBS from subjects that do not respond to said CAR therapy.

11. The method of claim 1, wherein response to said CAR therapy is complete response (CR).

12. The method of claim 1, wherein said PBS was produced from peripheral blood obtained from said subject at about day 5 after said CAR therapy.

13. The method of claim 12, wherein response to said CAR therapy is CR and said CR is CR at day 30 or beyond after said CAR therapy.

14. The method of claim 1, wherein a number of CAR cells in said PBS above a predetermined threshold indicates said subject is likely to suffer from an extended cytokine release syndrome (CRS) as compared to the average CRS duration in subjects receiving said CAR therapy.

15. The method of claim 14, wherein at least one of:
a. said predetermined threshold is the number of CAR cells in PBS from subjects without an extended CRS;
b. said PBS was produced from peripheral blood obtained from said subject at about day 14 after said CAR therapy; and
c. said number of CAR cells is above said predetermined threshold in PBS produced from peripheral blood obtained from said subject at least 3 time points between days 1 and 14 after said CAR therapy.

16. The method of claim 1, wherein the number of CAR cells is the total number of CAR cells.

17. The method of claim 1, wherein the number of CAR cells is the total number of activated CAR cells.

18. The method of claim 1, wherein the number of CAR cells is the total number of regular CAR cells.

19. The method of claim 1, further comprising administering said CAR therapy to said subject, obtaining peripheral blood from said subject and producing smears from said peripheral blood.

20. The method of claim 1, wherein said determining the number of CAR cells in said PBS comprises counting said CAR cells with a digital microscopy platform that employs machine learning to determine cell classes based on morphology.
